(19) United States
(12) Patent Application Publication
(10) Pub. No.: US 2010/0318166 A1
(43) Pub. Date: Dec. 16, 2010

(54) METHODS AND SYSTEMS FOR ANTI-THROMBOTIC INTRAVASCULAR IMPLANTABLE DEVICES

(76) Inventor: Terrance Ransbury, Chapel Hill, NC (US)

Correspondence Address: PATTERSON THUENTE CHRISTENSEN PEDERSEN, P.A., 4800 IDS CENTER, 80 SOUTH 8TH STREET, MINNEAPOLIS, MN, 55402-2100, US

(21) Appl. No.: 12/815,210

(22) Filed: Jun. 14, 2010

Related U.S. Application Data
(60) Provisional application No. 61/186,811, filed on Jun. 12, 2009.

(51) Int. Cl. A61N 1/05

(52) U.S. Cl. 607/122

(57) ABSTRACT

An implantable intravascular medical device, and methods related to the implantable intravascular device, includes a plurality of containers connected by flex couplers which are configured for implantation in such a manner as to improve vascular response and reduce thrombosis resulting from chronic implantation of the device.

### RELATED APPLICATIONS

This application claims priority to U.S. Provisional Patent Application No. 61/186,811, filed Jun. 12, 2009, which is hereby incorporated by reference. The present application is related to, but does not claim the benefit of, U.S. Pat. No. 7,617,007 and U.S. Published Application Nos. 2007/0265673, 2008/0147168, 2008/0154327, 2008/0167702, 2009/0163927, and 2009/0192579, the disclosures of which are hereby incorporated by reference.

### FIELD OF THE INVENTION

The present invention relates generally to intravascular implantable devices, and more particularly to solutions for long-term anti-thrombotic implantation of such intravascular devices.

### BACKGROUND OF THE INVENTION

Implantable medical devices such as pacemakers, defibrillators, and implantable cardioverter defibrillators (“ICDs”) have been successfully implanted in patients for years for treatment of heart rhythm conditions. Pacemakers are implanted to detect periods of bradycardia and deliver low energy electrical stimuli to increase the heart rate. ICDs are implanted in patients to cardiovert or defibrillate the heart by delivering high energy electrical stimuli to slow or reset the heart rate in the event a ventricular tachycardia (VT) or ventricular fibrillation (VF) is detected. Another type of implantable device detects an atrial fibrillation (AF) episode and delivers electrical stimuli to the atria to restore electrical coordination between the upper and lower chambers of the heart. Still another type of implantable device stores and delivers drug and/or gene therapies to treat a variety of conditions, including cardiac arrhythmias. The current generation for all of these implantable devices are typically can-shaped devices implanted under the skin that deliver therapy via leads that are implanted in the heart via the patient's vascular system.

Next generation implantable medical devices may take the form of elongated intravascular devices that are implanted within the patient's vascular system, instead of under the skin. Examples of these intravascular implantable devices are described, for example, in U.S. Pat. Nos. 7,082,336, 7,529,589 and 7,617,007, and U.S. Published Patent Application Nos. 2004/0249431 and 2008/0167702. These devices contain electric circuitry and/or electronic components that are hermetically sealed to prevent damage to the electronic components and the release of contaminants into the bloodstream. Due to the length of these intravascular implantable devices, which in some cases can be approximately 10-60 cm in length, the devices generally are designed to be flexible enough to move through the vasculature while being sufficiently rigid to protect the internal components and provide adequate column strength for implanting the devices.

An alternative intravascular implantable device is described in U.S. Pat. Nos. 7,519,424, 7,616,992 and 7,627,376, all to Medtronic. The implantable device described therein includes a tether portion extending from the device body. The tether includes an anchoring member, and the tether is configured to extend through a vascular wall to a suitable fixation site such as directly to the external wall of the vessel, or tissue external to the vessel. The device is intended to be implanted through an incision in the subclavian vein, and retained within the vasculature above the atrium of the heart. A lead is typically coupled to the device, and extends down into the heart. The devices described therein utilize rechargeable and/or replaceable batteries, which aids in reducing device size to the detriment of shortened service life before the battery needs to be replaced or recharged.

Implantation of any kind of intravascular medical device carries the risk of thrombus formation within the vessel, which can negatively impact blood flow. In extreme cases, thrombus formation can lead to occlusion of the vessel. Over 100 years ago, Virchow postulated that three main risk factors increase the likelihood of thrombosis. The Virchow Triad includes: stasis resulting in changes to the blood flow (turbulence), trauma to or pressure on the vessel wall, and hypercoaguability (alteration in the constituency of blood). Since then, numerous efforts both theoretical and experimental have been made to understand blood flow and thrombosis, particularly in the context of arteriosclerosis. See, e.g., Chandran, K. B., “CardioVascular Biomechanics” New York University Press (August 1992). While useful for theoretical and steady state simulations, current theoretical models for understanding thrombosis tend to be either too simple or too complex and lack the flexibility to simulate the even more complex fluid and biological interactions resulting from introduction of an implantable device into the vasculature.

When designing an implantable intravascular device it is desirable to decrease the Virchow factors as much as possible to decrease the likelihood of thrombosis while maintaining functionality of the device and ease of implantation. The challenge, however, is that there are no effective models for understanding the implications of a particular design for an intravascular implantable device on blood flow and thrombosis. Prior intravascular implantable device configurations and anchoring arrangements attempted to minimize the risk of thrombosis formation, but an opportunity for improvement still exists.

### SUMMARY OF THE INVENTION

An implantable intravascular medical device, and methods related to the implantable intravascular device, in accordance with the present invention includes a plurality of containers connected by flex couplers which are configured for implantation in such a manner as to improve vascular response and reduce thrombosis resulting from chronic implantation of the device. Significant reduction in thrombosis formation have been observed in long-term implants of devices according to the present invention in numerous mammals.

In one embodiment, an elongate intravascular device is provided. The device includes a plurality of rigid containers, each container operable to contain at least one electronic component wholly within that container. The device further includes a plurality of flexible couplers, each flexible coupler connecting two of the rigid containers to form an elongated chain of containers and flexible couplers. And the device includes a flexible portion on an inferior end of the device, wherein the device is configured such that one container segment is positioned to extend completely through the right atrium of the heart of a patient with the inferior end of the device positioned within the inferior vena cava of the patient when the device is implanted within the patient and anchored in a vessel superior to the heart.

In another embodiment, a method of implanting an intravascular device includes introducing the device into the vasculature of a patient, the device having a plurality of containers connected by flex couplers, and anchoring the device within the vasculature at a location superior to the heart. The device is configured such that, once anchoring of the device is completed, a single one of the plurality of containers spans across the right atrium of the heart of the patient with at least a portion of the device superior to the heart and at least a portion of the device inferior to the heart.

In another embodiment, a method for distributing an intravascular implantable device includes providing an intravascular implantable device having a plurality of containers connected by flex couplers, and providing instructions for implanting the device into the vasculature of a patient. The instructions include introducing the device into the vasculature of a patient, the device having a plurality of containers connected by flex couplers, and anchoring the device within the vasculature at a location superior to the heart. The device is configured such that, once anchoring of the device is completed, a single one of the plurality of containers spans across the right atrium of the heart of the patient with at least a portion of the device superior to the heart and at least a portion of the device inferior to the heart.

Aspects of the present invention comprises specific arrangements of a combined solution to flow, chemistry, biology and vascular response to overcome thrombosis formation for an intravascularly implanted medical device of at least 5 cm in length and a diameter of at least about 9 French (3 mm). The problem of thrombosis formation, especially as it pertains to an intravascularly implanted medical device, is complex and not easily modeled. A clinically acceptable outcome requires a system-level solution to the problem, and as such, the solutions presented herein may be less effective if not implemented together.

Various combinations of features and characteristics described herein may be desirable for an intravascularly implanted device anchored superior to the heart with the device body extending through the heart, an unanchored inferior end, and an inferior end that does not extend below the renal veins.

In general, the various features and characteristics described in the embodiments herein are directed to ensuring that the chronically implanted intravascular medical device has a smooth profile, is sufficiently flexible to follow the contours of the vasculature during implantation and in response to patient movement, and avoids excessive pressure on or contact with the inner vessel walls.

### BRIEF DESCRIPTION OF THE DRAWINGS

The invention may be more completely understood in consideration of the following detailed description of various embodiments of the invention in connection with the accompanying drawings, in which:

FIG. 1 is a perspective illustration depicting human cardiac anatomy.

FIG. 2 is a plan view of an implantable intravascular device according to an example embodiment of the present invention.

FIG. 3a is a partial view of an implantable intravascular device having a thick device coating.

FIG. 3b is a partial view similar to FIG. 3a of an implantable intravascular device having a reduced thickness device coating.

FIG. 4 is a partial view of a coupler according to an example embodiment of the present invention.

FIG. 5a is a perspective view of an implanted intravascular device according to an example embodiment of the present invention.

FIG. 5b is a close-up partial view of FIG. 5a.

FIG. 6 is a perspective view of an implanted intravascular device according to an example embodiment of the present invention.

FIG. 7a is a partial view of a prior art implanted intravascular device.

FIG. 7b is a partial view of a prior art implanted intravascular device

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE DRAWINGS

In the following detailed description of the present invention, numerous specific details are set forth in order to provide a thorough understanding of the present invention. However, one skilled in the art will recognize that the present invention may be practiced without these specific details. In other instances, well-known methods, procedures, and components have not been described in detail so as to not unnecessarily obscure aspects of the present invention.

The present invention describes intravascular electrophysiological systems that may be used for a variety of functions to treat cardiac arrhythmias with electrical stimulation. These functions include defibrillation, pacing, and/or cardioversion. In general, the elements of an intravascular implantable device 100 (referred to herein as “IID” or “device”) for electrophysiological therapy include at least one elongate device body 104 and typically, but optionally, at least one lead 108 coupled to the body which may be anchored or retained in the vasculature or within the heart. Alternatively, the one or more leads 108 may not be anchored or retained in the vasculature or within the heart, or IID 100 may be provided with no leads, such as for an embodiment of an intravascular implantable drug/gene therapy device. Device body 104 is comprised of one or more rigid enclosures 112 that are combined using interconnecting flexible couplers 114.

Additional general information pertaining to the construction, arrangement and function of IID's suitable for use in accordance with the present invention can be found in U.S. Pat. Nos. 7,082,336, 7,363,082 and 7,529,589, and in U.S. Published Patent Application Nos. 2005/0228471, 2007/0265673, 2008/0147168, 2008/0154327 and 2008/0167702, the disclosures of which are hereby incorporated by reference in their entireties.

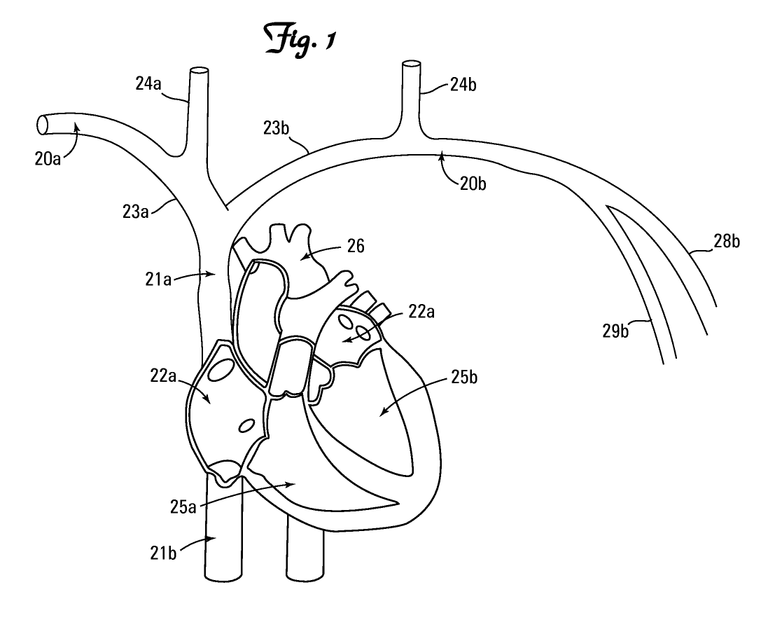

Referring now to FIG. 1, the general cardiac anatomy of a human is depicted, including the heart and major vessels. The following anatomic locations are shown and identified by the listed reference numerals: Right Subclavian 20a, Left Subclavian 20b, Superior Vena Cava (SVC) 21a, Inferior Vena Cava (IVC) 21b, Right Atrium (RA) 22a, Left Atrium (LA) 22b, Right Innominate/Brachiocephalic Vein 23a, Left Innominate/Brachiocephalic Vein 23b, Right Internal Jugular Vein 24a, Left Internal Jugular Vein 24b, Right Ventricle (RV) 25a, Left Ventricle (LV) 25b, Aortic Arch 26, Descending Aorta 27, Right Cephalic Vein 28a (not shown in FIG. 1), Left Cephalic Vein 28b, Right Axillary Vein 29a (not shown in FIG. 1) and Left Axillary Vein 29b.

Referring generally to the Figures, the IID 100 includes components known in the art to be necessary to carry out the system functions of an implantable intravascular electrophysiology device. For example, IID 100 may include one or more pulse generators, including associated batteries, capacitors, microprocessors, communication circuitry and circuitry for generating electrophysiological pulses for defibrillation, cardioversion and/or pacing. IID 100 may also include detection circuitry for detecting arrhythmias or other abnormal activity of the heart. The specific components to be provided in IID 100 will depend upon the application for the device, and specifically whether IID 100 is intended to perform defibrillation, cardioversion, and/or pacing along with sensing functions.

In one embodiment, IID 100 can have a streamlined maximum cross sectional diameter which can be in the range of 3-15 mm or less, with a maximum cross-sectional diameter of 3-8 mm or less in one embodiment. The cross-sectional area of IID 100 in the transverse direction (i.e. transecting the longitudinal axis) can preferably be as small as possible while still accommodating the required components. This area can be in the range of approximately 79 mm̂2 or less, in the range of approximately 40 mm̂2 or less, or between 12.5-40 mm̂2, depending upon the embodiment and/or application.

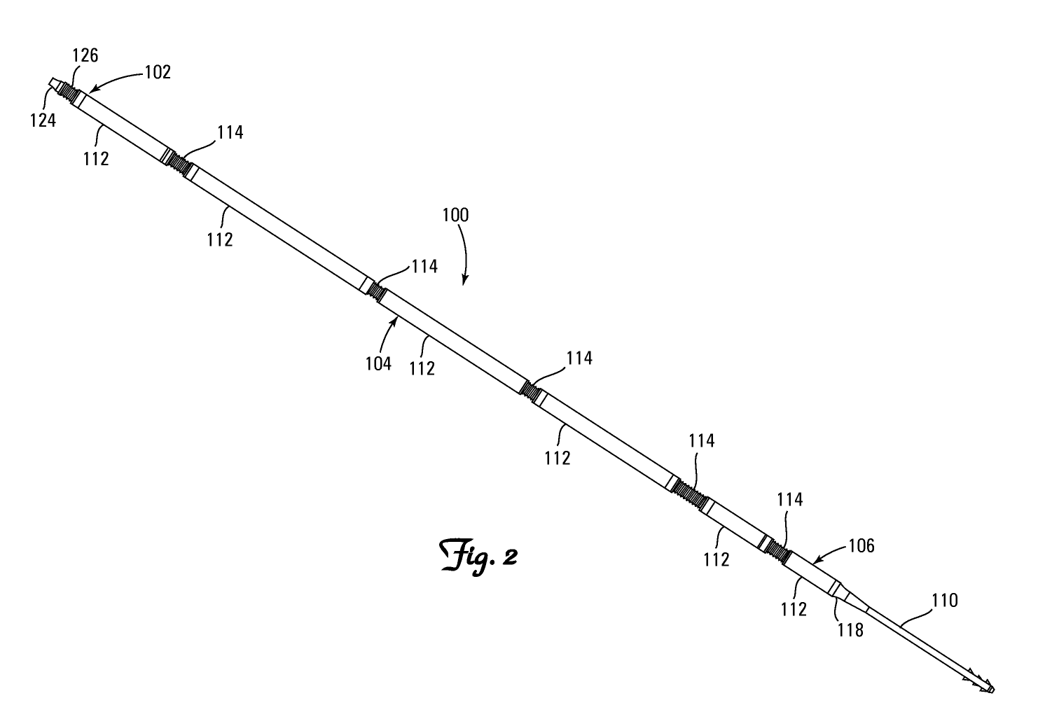

Referring to FIG. 2, device body 104 is comprised of one or more rigid containers 112, or segments, which house the necessary components. These containers 112 can be of any appropriate shape, cross-section, and length, but are depicted herein as having a cylindrical shape with a diameter of approximately 3-15 mm and a length of approximately 20 mm to 75 mm. In order to allow for insertion of the device body 104 comprising rigid containers 112 into the vasculature, it can be desirable to limit the diameter to less than about 8 mm with a length of no more than about 70 cm. Given the minimal space allowed for components, it can be desirable to arrange the device components within containers 112 so as to make efficient use of the available space. The length of individual containers 112 can vary, depending upon the ultimate destination of each container within the vasculature, and the path through which each component must pass, as the amount of bending and varying size of the path can affect the maximum container size for different areas of the vasculature.

The thickness of the walls of containers 112 also can vary, depending upon the application and the material being used. It can be desirable for the walls to be as lightweight as possible, while still providing for sufficient rigidity. In one example, container 112 can be made of a biocompatible material that is capable of sterilization and is conductive, with a sidewall thickness on the order of about 0.001″ to 0.005″. Possible materials include titanium, nitinol, stainless steel, nickel, or alloys thereof, as well as polymers such as nylon or polyurethane. The sidewall thickness can vary among containers 112, as well as within an individual container 112 in order to accommodate the internal components, etc.

Any desired number of containers 112 can be combined using interconnecting flexible couplers 114 (or “bellows”) in order to form a flexible device, such as in FIGS. 2 and 5a-6. For many devices, this will include a string of at least three containers 112. Coupler 114 may comprise a plurality of convolutes, folds or joints 115. The overall length of coupler 114 may be modified by including greater or fewer joints 115 in coupler 114, or adjusting the size or spacing of joints 115. In one embodiment, coupler 114 includes at least four joints 115. In another embodiment, coupler 114 includes at least eight joints 115. In a further embodiment, coupler 114 includes at twelve joints 115. In other embodiments, coupler 114 may include up to fifty joints 115, or greater than fifty if desired. The couplers 114 can be of any appropriate shape, but can preferably have a shape similar in cross-section to the cross-section of the container, in order to prevent the occurrence of edges or ridges that can give rise to problems such as the formation of blood clots in the vasculature. Couplers 114 can be made of a biocompatible material similar to the containers 112. A connecting process such as welding can be used to form a continuous, hermetic seal between couplers 114 and containers 112. Couplers 114 feature an internal passage 117 for the routing of electronic or fiberoptic cabling, wiring, or pressure vessels configured to transmit signals and/or power between components in separate containers 112.

In general, it is desirable for the overall shape and configuration of device 100 to present a smooth profile, lacking any sharp edges, blunt faces or abrupt changes in profile. In addition to reducing flow disturbance, a smooth finish aids device 100 in sliding along the vessel wall as needed during the implant procedure and chronic implantation, with minimal force and potential for damage to the endothelial layer. As such, a substantially isodiametric profile for device 100 is desirable, and various coatings and/or overmoldings may be applied to lead 108, tip portion 110, containers 112, and/or bellows 114 to smooth the overall profile of IID 100.

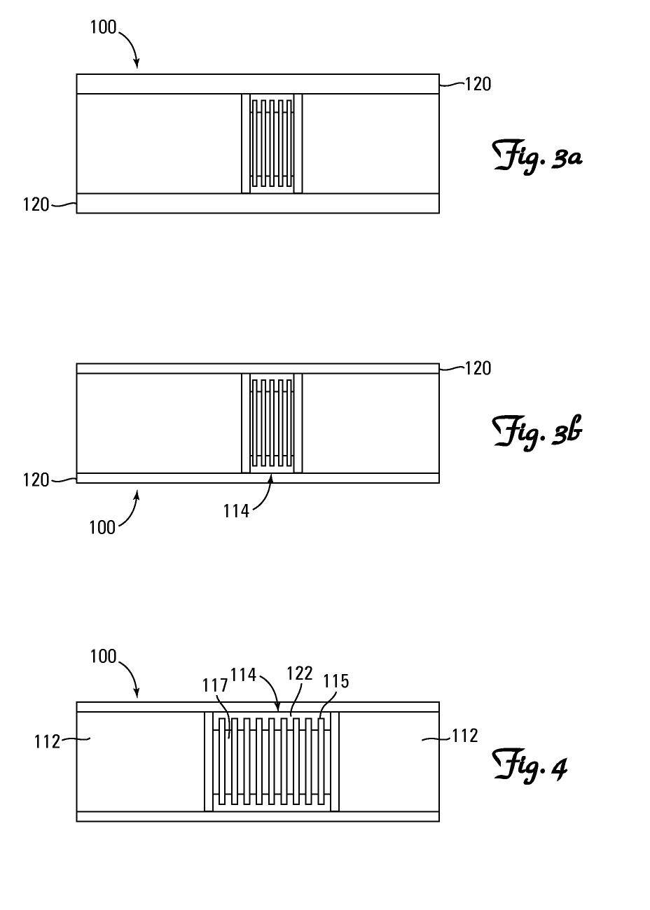

Containers 112 may be covered by a layer or coating 120, such as depicted in FIG. 3b, which may be electrically insulative, particularly if the container material is conductive. One example of such a coating is ePTFE. It is also desirable to provide a coating 120 that is anti-thrombogenic (e.g., perfluorocarbon coatings applied using supercritical carbon dioxide) so as to prevent thrombus formation on the device 100. It also can be beneficial for coating 120 to have anti-proliferative properties so as to minimize endothelialization or cellular ingrowth, since minimizing growth into or onto device 100 can help minimize vascular trauma in the event device 100 is ever is explanted. Coating 120 may also be selected to elute anti-thrombogenic compositions (e.g., heparin sulfate) and/or compositions that inhibit cellular in-growth and/or immunosuppressive agents. If the containers 112 are conductive, coating 120 may be selectively applied or removed to leave an exposed electrode region 116 on the surface of device 100 where necessary. Electrode region 116 may be configured to deliver therapy and/or perform one or more sensing functions, and the transition between exposed electrode region 116 and the neighboring surface area should be made as smooth as possible.

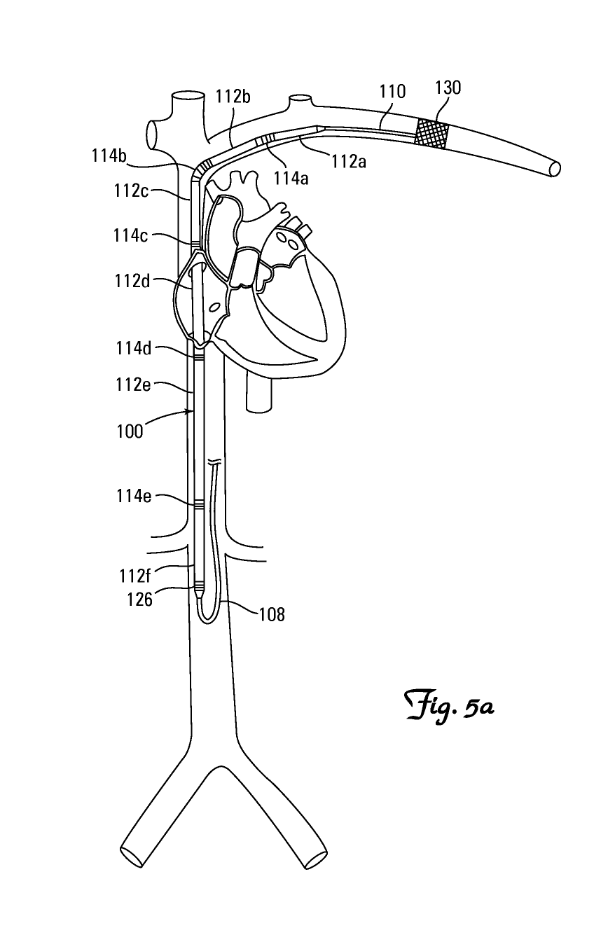

An overmold booting 122 of a material such as silicone or polyurethane can be formed around the bellows 114 to provide rigidity and column strength, as depicted in FIG. 4. An overmold sleeving 122 can decrease the flexibility of the device where more rigidity is desired. An overmold sleeving also can be more flexible than the bellows such that the bellows are the primary limiting factor on flexibility. The overmold 122 also can function to create an isodiametric junction between bellows 114 and containers 112, to prevent the occurrence of ridges, edges, and valleys that could otherwise be present on the outside surface of the 114 bellows. Making the outer surface of couplers 114 relatively smooth prevents the occurrence of turbulence and clotting of the blood that could otherwise result from a rippled surface. Coating 120 may also be applied over couplers 114, or over any booting 122 on couplers 114. Welded junctions between metal components, such as between container 112 and bellows 114, may be ground or polished smooth, and/or covered with a suitable coating as described herein.

Referring to FIGS. 2, 5a and 6, tip portion 110 on distal end 106 is coupled to device body 104, and is configured to mate with a suitable anchor arrangement 130. Tip portion 110 is sufficiently flexible to allow bending during implantation yet is preferably more rigid than, for example, a conventional cardiac lead body. Flexibility of tip portion 110 further aids in permitting overall device movement during chronic implantation in response to skeletal, cardiac, or respiratory movements and gravitational effects. Various flexible bio-compatible materials such as Elasteon or silicone, or other materials known in the art may be utilized for tip portion 110. Tip portion 110 may include an internal cable (not shown), to provide axial tensile strength to the bio-compatible material. Tip portion 110 may comprise a smaller diameter than device body 104, and include a transition portion 118 that presents a smooth taper between the differing diameters.

A similar transition portion 124 may be provided at the junction between lead 108 and device 100. Other than the transition portion 124, the distal portion of lead 108 may be similar to a conventional defibrillation/pacing lead, and generally includes one or more conductors and electrodes in an elongated, sealed and insulated protective structure that is adapted to withstand chronic implantation. Conventional defibrillators and pacemakers, which are implanted in a subcutaneous pocket in the chest of the patient, feature one or more leads routed through the subclavian vein, onto the superior vena cava, and finally into the heart via the right atrium. In one embodiment, lead 108 is provided on the proximal/inferior end of device 100, and must be routed from the inferior vena cava up to the right atrium and into the heart. As such, lead 108 must be sufficiently flexible and pliable so as to not impart significant pressure on the vessel wall.

Anchor 130 is configured to retain device 100 within a patient's vasculature, and in one embodiment anchor 130 comprises a conventional intravascular stent. In one embodiment, the device is anchored superior to the heart, such as in the subclavian vein, brachiocephalic vein, or jugular vein. A suitable anchor arrangement provides stability to the device within the vessel and provides sufficient radial strength to maintain vessel patency. Once deployed, anchor 130 can be intimate to the vessel wall, which is distended slightly, allowing the vessel lumen to remain approximately continuous despite the presence of the anchor and thus minimizing turbulence or flow obstruction. In another embodiment, anchor 130 may be integrated with device 100, such as disclosed in U.S. Provisional Application No. 61/186,811, filed Jun. 12, 2009, the disclosure of which is incorporated by reference.

Device 100 can be implanted using any of a number of implantation techniques, such as over a guidewire, with the use of a pusher, or with the use of a grasper to guide the device through the vasculature. Suitable sites for introduction of the IID 100 into the body can include, but are not limited to, the venous system using access through the right or left femoral vein or the right or left subclavian vein. The ability of the bellows couplers 114 to flex, combined with the lateral strength of the interconnections, allows for some degree of steering of the device during implantation into a patient's body. In one embodiment, the bellows 114 provides 1:1 torque transmission over the entire length of the overall device 100. This torque transmission capability, when combined with the curvature can provide steerability when navigating the device into place within a patient's body.

While increasing the overall flexibility of device 100 is desirable for obtaining favorable vascular response in a chronic implant, that desire must be weighed against a minimum required amount of rigidity (in the form of column strength of the device) necessary for delivery of the device during implantation. Although device 100 may be implanted in numerous ways, most involve the device being pushed in some manner. If device 100 lacks sufficient column strength, the device will “jack knife” within the vasculature, making it nearly impossible to deliver the device to the desired anchoring location, especially if the desired anchor location is superior to the heart.

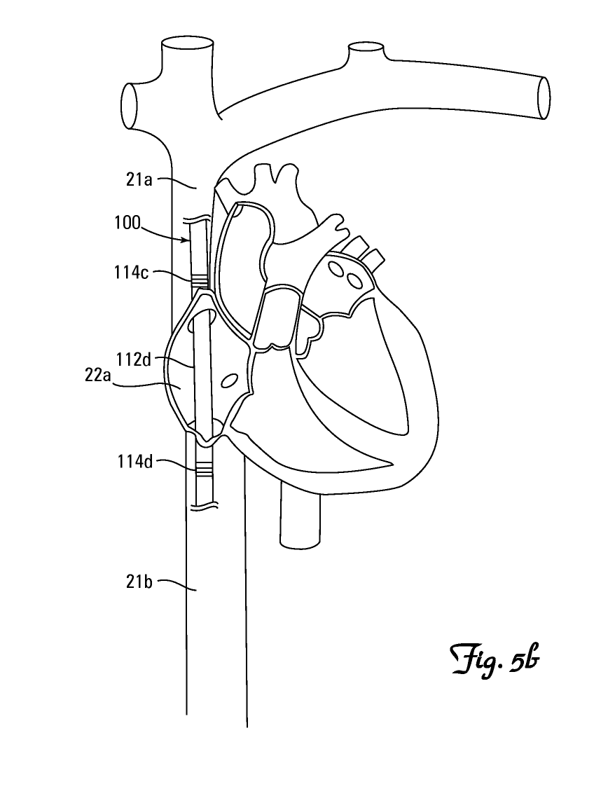

Therefore, it has been found that increasing flexibility only of select portions of device 100 is more effective at obtaining favorable vascular response, while maintaining sufficient column strength to aid in implantation. Referring to FIG. 5a, device 100 in one embodiment comprises a plurality of containers 112a, 112b, 112c, 112d, 112e, and 112f, respectively connected by couplers 114a, 114b, 114c, 114d and 114e. Device 100 is secured within the vasculature with anchor 130 at tip portion 110, and features a lead 108 located at proximal/inferior end 102 adjacent to flex portion 126.

As depicted in FIG. 5a, coupler 114b is illustrated as being longer than other similar couplers. While seemingly counterintuitive to increase the length of coupler 114b and thereby increase overall length of device 100, the benefit is an improvement in vascular response. In another embodiment, coupler 114b may be of a similar length to couplers 114a and 114c-e, but include a lower durometer overmold 122 and/or a thinner coating 120 than other couplers, so as to be comparatively more flexible.

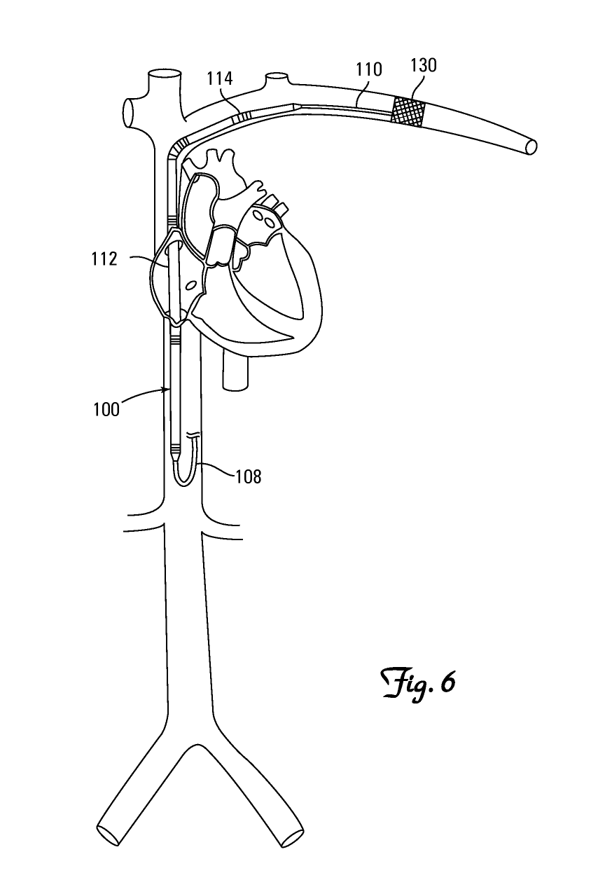

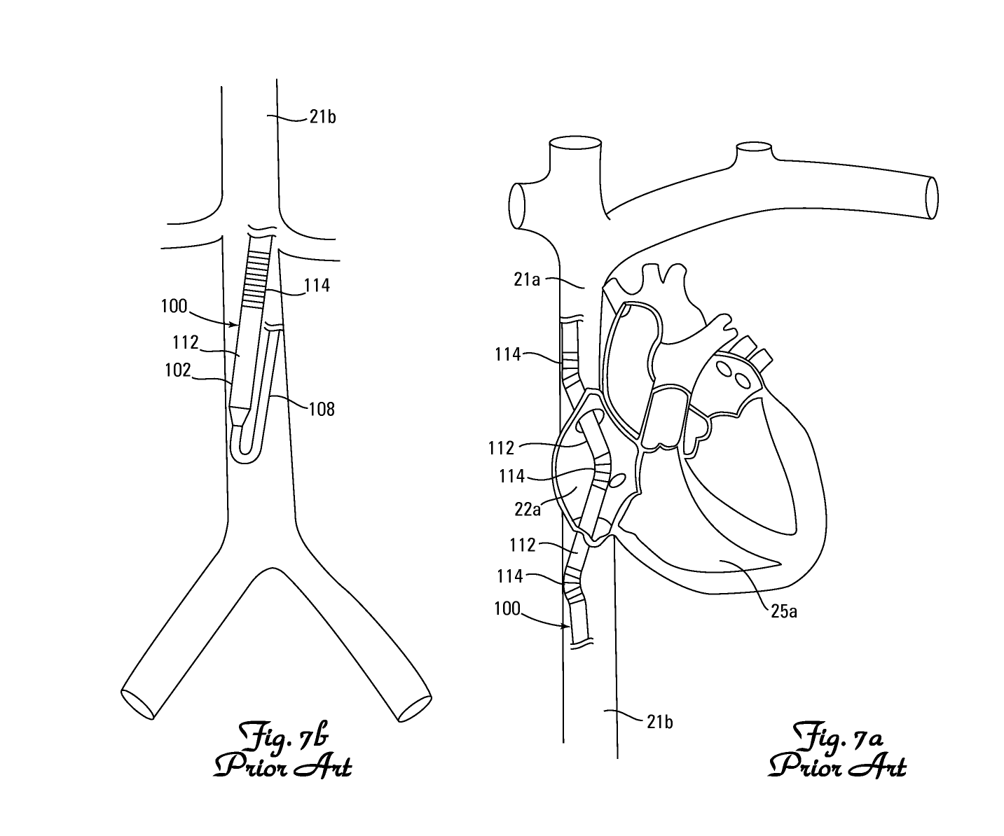

Containers 112a-f are depicted as having varying sizes. While a single size container used throughout device 100 may be advantageous from a manufacturing standpoint, it is not necessarily advantageous for reducing thrombosis in a chronic implant. As depicted in FIGS. 5a-6, container 112d is configured to span across the atrium of the heart. Clinical experiments have shown that maintaining rigidity of the portion of device 100 that resides within the atrium allows device 100 to remain as parallel as possible to the vasculature. This arrangement reduces any pressure applied on the vessel wall by device 100, and reduction in pressure against the vessel wall is paramount to minimizing thrombosis. If the device is configured such that when implanted, a coupler is positioned within the atrium, the pumping of the heart will tend to suck the device toward the right ventricle, thereby deforming the device and pulling other container segments into the vessel wall near the heart, as depicted in FIG. 7a.

Flex portion 126 is positioned at proximal end 102 of device body 104. Flex portion/segment 126 may be constructed similarly to bellows 114 as disclosed herein, or may comprise a suitable polymer or other segment of flexible material. The added flexibility from flex portion 126 reduces any force on the vessel wall at the proximal end of device 100, especially when dealing with the tortuosity of the vessel and the potential for damage due to motion of the device, both laterally and vertically, within the vessel. In one embodiment, flex portion 126 provided on the proximal (inferior) end of the device is generally of the same diameter as that of the device body, but with much greater flexibility. While negatively impacting the overall length of device 100, the addition of flex portion 126 reduces pressure against the vessel wall caused by wagging of the device 100 within the vasculature, such as in FIG. 7b.

A number of experiments were carried out to study the flexibility of the implantable intravascular device, and reduce thrombosis formation. Previous embodiments of the implantable intravascular device included a coating 120 having a thickness of approximately 0.0084 inches, and a bellows overmold 122 having a durometer of approximately 70. By reducing the thickness of coating 120 to approximately 0.004 inches, and applying overmold material 120 having a durometer of approximately 30, a significant reduction in thrombosis formation and therefore improvement in vascular response was achieved. Further, initial embodiments of flex portion 126 had a bending force of 0.49 lbs, that was determined to be too stiff to provide satisfactory resistance to thrombosis. Subsequent embodiments of flex portion 126 were constructed with a bending force of only 0.167 lbs, with superior results. The reduction in stiffness of flex portion 126 was achieved by modifying characteristics of coating 120 and overmold 122.

In one embodiment depicted in FIG. 6, IID 100 may have an overall length such that when implanted and anchored superior to the heart, the proximal end of the device extends into inferior vena cava but remains above the junction with the renal veins. It is believed that most of the improved features and characteristics described herein are necessary for such an embodiment, with device 100 anchored superior to the heart, device body 104 extending through the heart, and an unanchored inferior end 102 that does not extend below the renal veins.

In another embodiment depicted in FIG. 5a, IID 100 may have an overall length such that when implanted and anchored superior to the heart, the proximal end of the device extends into inferior vena cava and below the junction with the renal veins. It is believed that all of the improved features and characteristics described herein are necessary for such an embodiment, with device 100 anchored superior to the heart, device body 104 extending through the heart, and an unanchored inferior end that extends below the renal veins.

In one embodiment, instructions for implanting IID 100 in accordance with the various embodiments described herein in the form of printed or electronically, optically or magnetically stored information to be displayed, for example, are provided as part of a kit or assemblage of items prior to surgical implantation of device 100. In another embodiment, instructions for implanting device 100 in accordance with the various embodiments described herein are provided, for example, by a manufacturer or supplier of IID 100 separately from providing the device, such as by way of information that is accessible using the Internet or by way of seminars, lectures, training sessions or the like.

Various embodiments of systems, devices and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

It should be pointed out that many of the retention devices and methods, implantation methods and other features are equally suitable for use with other forms of intravascular implants. Such implants might include, for example, implantable neurostimulators, artificial pancreas implants, diagnostic implants with sensors that gather data such as properties of the patient's blood (e.g. blood glucose level) and/or devices that deliver drugs or other therapies into the blood from within a blood vessel.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the various embodiments of the present invention, it is expressly intended that the provisions of Section 112, sixth paragraph of 35 U.S.C. are not to be invoked unless the specific terms “means for” or “step for” are recited in a claim.

1. An elongate intravascular device, comprising:
a plurality of rigid containers, each container operable to contain at least one electronic component wholly within that container;
a plurality of flexible couplers, each flexible coupler connecting two of the rigid containers to form an elongated chain of containers and flexible couplers; and
a flexible portion on an inferior end of the device,
wherein the device is configured such that one container segment is positioned to extend completely through the right atrium of the heart of a patient with the inferior end of the device positioned within the inferior vena cava of the patient when the device is implanted within the patient and anchored in a vessel superior to the heart.

2. The device of claim 1, further comprising:
a lead coupled to the flexible portion, such that the flexible portion is between the lead and the inferior end of the device.

3. The device of claim 1, wherein the flexible portion is at least 50% more flexible than the plurality of flexible couplers.

4. The device of claim 1, wherein the flexible portion is of a greater length than the plurality of flexible couplers.

5. The device of claim 1 wherein the flexible portion is one of the plurality of flexible couplers.

6. The device of claim 5, wherein the flexible portion is at least 50% more flexible than the other ones of the plurality of flexible couplers.

7. The device of claim 5, wherein the flexible portion is of a greater length than the other ones of the plurality of flexible couplers.

8. The device of claim 2 wherein the lead has a length and a flexibility that is configured to permit the lead to extend inferior from the flexible portion and then bend back to have a distal end positioned superior to the flexible portion.

9. The device of claim 1 wherein the flexible portion has a flexibility such that the flexible portion exerts no more than about 0.25 pounds of force against a wall of the inferior vena cava.

10. The device of claim 2 wherein the flexible portion has a flexibility such that the lead exerts no more than about 0.25 pounds of force against a wall of the inferior vena cava.

11. The device of claim 1 wherein has a smooth exterior profile and is sufficiently flexible to follow contours of the vasculature of the patient during implantation and in response to patient movement.

12. A method of implanting an intravascular device, comprising:
introducing the device into the vasculature of a patient, the device having a plurality of containers connected by flex couplers; and
anchoring the device within the vasculature at a location superior to the heart,
the device being configured such that, once anchoring of the device is completed, a single one of the plurality of containers spans across the right atrium of the heart of the patient with at least a portion of the device superior to the heart and at least a portion of the device inferior to the heart.

13. The method of claim 12 wherein the device includes a lead extending from a flexible portion proximate an inferior end of the device and the method further comprises:
implanting a distal portion of the lead at a location superior to the inferior end of the device.

14. The method of claim 13 where the flexible portion and the lead are configured such that the flexible and the lead exert less than 0.25 pounds of force against a wall of the inferior vena cava after implanting the distal portion of the lead.

15. A method for distributing an intravascular implantable device, comprising:
providing an intravascular implantable device having a plurality of containers connected by flex couplers; and
providing instructions for implanting the device into the vasculature of a patient, the instructions including:
introducing the device into the vasculature of a patient, the device having a plurality of containers connected by flex couplers; and
anchoring the device within the vasculature at a location superior to the heart,
the device being configured such that, once anchoring of the device is completed, a single one of the plurality of containers spans across the right atrium of the heart of the patient with at least a portion of the device superior to the heart and at least a portion of the device inferior to the heart.

16. The method of claim 15 wherein the device includes a lead extending from a flexible portion proximate an inferior end of the device and the method further comprises providing instructions for:
implanting a distal portion of the lead at a location superior to the inferior end of the device.

17. The method of claim 15 where the flexible portion and the lead are configured such that the flexible and the lead exert less than 0.25 pounds of force against a wall of the inferior vena cava after implanting the distal portion of the lead.
